(19) United States
(12) Patent Application Publication
(10) Pub. No.: US 2026/0240828 A1
(43) Pub. Date: Aug. 20, 2026

(54) GLYCOSPHINGOLIPID BIOSYNTHESIS INHIBITORS FOR TREATMENT OF TUMOURS DEPENDENT ON THE ONCOGENE GOLPH3 AND ON ONCOGENES OF THE GOLPH3 CLUSTER

(71) Applicant: Golgenia S.R.L., Milano (IT)

(72) Inventor: Alberto Luini, Milano (IT)

(73) Assignee: Golgenia S.R.L., Milano (IT)

(21) Appl. No.: 19/160,516

(22) PCT Filed: Feb. 28, 2024
(86) PCT No.: PCT/IB2024/051906

§ 371 Date: Aug. 28, 2025

(30) Foreign Application Priority Data
Mar. 2, 2023 (IT) 102023000003750

(51) Int. Cl. A61K 31/445; A61K 31/4025; A61K 31/496; A61P 35/00; C12Q 1/6886

(52) U.S. Cl. CPC A61K 31/445; A61K 31/4025; A61K 31/496; A61P 35/00; C12Q 1/6886; C12Q 2600/158

(57) ABSTRACT

The object of the present invention is the use of glycosphingolipid biosynthesis inhibitors for the treatment of tumours dependent on the oncogene GOLPH3 and on two oncogenes similar to GOLPH3 (GOLPH3-L and LCS), which form the GOLPH3 cluster with GOLPH3. A further object of the invention is the use of detection of genomic amplification of GOLPH3 as predictive marker for identification of tumours that respond to said inhibitors.

The object of the present invention is the use of glycosphingolipid biosynthesis inhibitors for the treatment of tumours dependent on the oncogene GOLPH3 and on two oncogenes similar to GOLPH3 (GOLPH3-L and LCS), which form the GOLPH3 cluster with GOLPH3. A further object of the invention is the use of Golph3 as marker for identification of tumours sensitive to said inhibitors.

### PRIOR ART

Golgi phosphoprotein 3 (GOLPH3) is an oncoprotein encoded by a gene found in an area of the human genome which is often amplified in various solid tumours, including melanoma, lung cancer, breast cancer, glioma and colorectal cancer (Rizzo R et al., Tissue Cell 2017; 49 (2 Pt A): 170-174). The reported frequency varies according to the source (databases or publications). For example, according to an authoritative, conservative database, the amplification frequency of GOLPH3 is 11% in squamous-cell lung cancer, 7% in pulmonary adenocarcinoma and 6% in carcinoma of the uterus (The Cancer Genome Atlas).

GOLPH3 is not only genomically amplified but also overexpressed (in a larger number of tumours). Said overexpression is correlated with an unfavourable prognosis in several types of tumour, including 52% of breast cancers and 41% to 53% of glioblastomas (Sechi S et al. Int. J. Mol. Sci. 2020; 21:933). Said percentages also vary according to source (databases or publications). Finally, the oncogene GOLPH3 confers chemoresistance, a serious obstacle to pharmacological treatment of tumours.

The oncogenic action mechanism of GOLPH3 (in particular as regards its chemoresistance-inducing properties) has been suggested to be due to said oncogene's property of selectively stimulating transport of growth receptors to the plasma membrane, and thus activating said receptors (Farber-Katz S E et al. Cell. 2014 156 (3): 413-27; Buschman M D et al. Cancer Res. 2015 75 (4): 624-7). In agreement with said finding, other authors then demonstrated that GOLPH3 acts by activating signalling pathways like those initiated by growth factor receptors (such as the EGF receptor) or those mediated by oncogenic kinases and GTPases such as PI3K kinase, AKT, mTOR, RAS, etc. (Zhi-Ping Zhou et al. Int J Oncol. 2018 53 (3): 1183-1192; Ming-Zhen Wang Molecular Medicine Reports 2018 17:542-548).

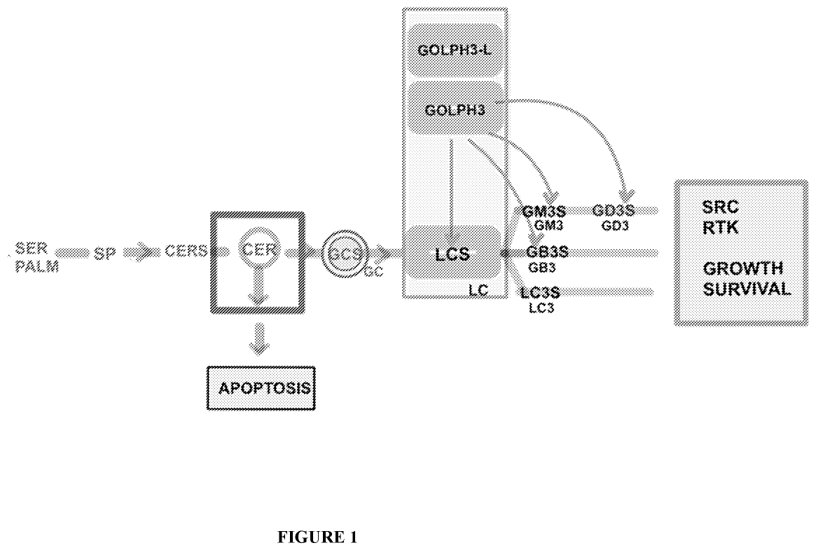

More recently, the oncogenic effect of GOLPH 3 was suggested to be connected with glycosphingolipid (GSL) metabolism by Rizzo R et al. in EMBO J. 2021:15; 40 (8). Said authors demonstrated that GOLPH3 acts by increasing the levels, and therefore the activity, of a group of GSL synthesis enzymes, namely (in the order wherein they act) lactosylceramide synthase (LCS), GB3 synthase (GB3S), GM3 synthase (GM3S) and GD3 synthase (GD3S), which therefore increase the amount of their metabolic products (LC, GB3, GM3, GD3) at the expense of their precursors, glucosylceramide (GC) and ceramide (Cer) (scheme in FIG. 1).

Said metabolites are bioactive; ceramide is a tumour suppressor, inhibits growth and induces apoptosis, whereas GB3 and GD3 activate SRC and growth factor receptors, which stimulate cell growth. The result of reduced ceramide and increased GB3 and GD3 is therefore inhibition of apoptosis and strengthening of proliferative stimuli. Said effects can protect the tumour cell against stress-induced ceramide production (see below) and stimulate cell proliferation, thereby potentially contributing to tumour development. The other oncogenes belonging to the GOLPH3 cluster, GOLPH3-L and LCS, have very similar effects to those induced by GOLPH3 (FIG. 1). The metabolic pathway of GSL and the action mechanism of GOLPH3 are illustrated in FIG. 1.

A series of synthetic reactions (presented in the horizontal sequence) starting from serine (SER) and palmitic acid (PALM) produces sphingosine (SP), which is then used by ceramide synthases (CerS) to form Cer. Cer (black square) is a tumour suppressor, and is functionally the key molecule in the system because its formation induces apoptosis, while its consumption produces cell proliferation. Cer is consumed and converted in the Golgi complex into glucosylceramide (GC) by glucosylceramide synthase (GCS, drug target, in the concentric circles). GC is then transferred from the cytosolic leaflet to the luminal leaflet of the Golgi membrane by the action of a “flippase”, P-glycoprotein (P-gp, which is also one of the molecular “pumps” known to extrude various molecules, including medicaments, from the cell).

In the lumen, GC is then converted to lactosylceramide (LacCer) by LCS. LacCer acts as substrate for the synthesis of all the complex GSLs, including GB3 and globosides, GM3 and gangliosides and LC3 and lactosides. The action mechanism of the GOLPH3 cluster is shown in the central vertical rectangle. GOLPH3 (against the grey background) is a GSL metabolism regulator. It acts on the enzymes mentioned in the text (arrows), increasing their levels and consequently their activity. Said increase has two complementary effects: a) consumption of Cer, and therefore weakening of apoptosis, and b) increased formation of the products of said enzymes which have stimulating effects on SRC and on growth factor receptors (RTK), namely stimulation of cell proliferation. As regards the other oncogenes belonging to the GOLPH3 cluster, GOLPH3-L (grey background, top) is very similar to GOLPH3, and has the same effect, while LCS (grey background, top) is the main mediator of the effect of GOLPH3, and has the same effect as GOLPH3.

The role of GSL in tumours as markers and cell-signal regulating molecules was described by Furukawa et al. in Cancer Sci., 2019; 110:1544-1551 and Ogretmen B in Nature Reviews, 2018; 18:33-50. The possibility of counteracting tumour growth by inhibiting GSL biosynthesis with glucosylceramide synthase (GCS) inhibitors has been studied for some time; for example, Inokuchi et al., Cancer Letters, 38, 1987, 23-30, described the anti-tumoral activity of 1-phenyl-2-decanoylamino-3-morpholine propanol (PDMD), a UDP-glucose ceramide glucosyltransferase inhibitor, in a murine tumour model (Ehrlich ascites).

Stefanovic et al., in Oncotarget, 2016; 7 (7): 8254-8263 reported the ability of some GSL biosynthesis inhibitors, especially GCS inhibitors (PDMD, imipramine) to improve the activity of sorafenib in vitro and in vivo against hepatocellular carcinoma cells. Tyler et al., Experimental Cell Research, 2015; 3 3 6:23-32, described potentiated cisplatin cytotoxicity by PDMD on melanoma cells and pleural mesothelioma. The potential anti-tumoral activity of GCS inhibitors belonging to the chemical class of iminosugars such as miglustat and derivatives thereof in Lovo and HCT116 cells and in murine models of colorectal adenoma was described in Int. J. Mol. Sci. 2021, 22, 10539; or those belonging to the class of ceramide analogues such as PDMP and eliglustat; or the class of P-gp inhibitors such as zosuquidar and analogues thereof (Gottesman M M et al. FEBS Lett. 2006 13; 580 (4): 998-1009) or such as the tamoxifen metabolite 4-hydroxytamoxifen (Morad S A F et al. Biochim Biophys Acta. 2015; 1851 (9): 1134-1145.).

However, despite some positive data, the cases of proven efficacy of GSL metabolism inhibitors used as antitumorals in cell and animal models examined to date are rare and unpredictable. Most researchers have published data regarding experimental conditions and the cell lines wherein the effects of GSL metabolism inhibition are documentable but not, as is often the case in research, data regarding cases wherein said inhibitors have no effect.

It has now been found, by comparing the effect of GSL metabolism inhibitors on tumour cells characterised by amplification of GOLPH3 and genes belonging to the GOLPH3 cluster with the effect of said inhibitors in tumour cells not exhibiting said amplification, that only a limited proportion of tumour lines, namely those characterised by GOLPH3 amplification (for example, as already stated, 10% in squamous-cell lung cancer lines) or by strong overexpression of genes belonging to the GOLPH3 cluster, is repeatably and predictably sensitive to GSL metabolism inhibitors in both cell growth assays and stress resistance assays, such as that due to DNA damage caused by chemotherapy.

This agrees with the findings reported in the DEPMAP database (The Cancer Dependency Map Program at the Broad Institute), which demonstrate that GCS depletion significantly inhibits the growth of 2 out of 100 tumour lines grown under adhesion to the cell matrix in vitro. Said success rates are far from being clinically useful. An antitumoral action by said inhibitors has never been demonstrated in clinical practice. Only for miglustat and derivatives thereof has efficacy in reducing activation of the osteoclasts and bone destruction associated with multiple myeloma been reported (J Clin Invest. 2015; 125 (6): 2279-2292). However, the effect described is limited to osteoclasts.

On the whole, said data indicate that only the proportion of tumours characterised by amplification of GOLPH3 and the GOLPH 3 Cluster is sensitive to the effect of GSL biosynthesis inhibitors, and thus that only in said tumours do glycosphingolipid synthesis inhibitors have any prospect of pharmacological use in a clinical setting. This is a common situation in the development of many targeted medicaments.

It should be noted that it was not predictable before the study presented here that GSL metabolism inhibitors were systematically and predictably active only in tumour cells with amplification of GOLPH3. In fact, glycosphingolipid metabolism has the potential to activate mitogenic signalling in all mammal cells. The reason why only tumours with amplified GOLPH3 respond is probably that Golph3 is a very powerful activator of said metabolic pathway, because it simultaneously activates different enzymes of said metabolism (see FIG. 1), and has therefore been adopted as a defence mechanism by various types of tumour.

### DESCRIPTION OF THE INVENTION

It has now been discovered that GSL biosynthesis inhibitors (scheme in FIG. 1) are selectively useful in the treatment of tumours dependent on the GOLPH3 cluster of oncogenes. Specifically, it has been found that GSL biosynthesis inhibitors only inhibit the growth of tumour lines if the tumour lines treated with inhibitors 1) express at least one of the oncogenes belonging to the GOLPH3 cluster, and 2) grow under stress conditions due to the presence of chemotherapy agents or loss of cell-matrix anchorage. Conversely, said inhibitors have little influence on the growth of the same tumour lines under growth conditions of adhesion to the matrix in the absence of stress.

These observations indicate that 1) the presence of an oncogene belonging to the GOLPH3 cluster can be used as a predictive marker to identify tumours sensitive to inhibition of GSL metabolism, and 2) the inhibitors only act in said tumour lines under stress conditions, abolishing both their chemoresistance and their resistance to loss of anchorage to the matrix, which normally induces apoptosis. Both of said conditions are of considerable clinical importance. Chemoresistance is one of the major problems involved in cancer treatment, and resistance to loss of anchorage is known to reflect the ability of the tumour to grow in vivo and metastasise.

GCS inhibitors already used in clinical practice to treat other disorders (genetic disorders involving lysosomal accumulation) have been used as GSL metabolism inhibitors, as have P-GP inhibitors, which are also GSL metabolism inhibitors. This strategy therefore involves repositioning GSL metabolism inhibitors for antitumoral use, which enables expensive development steps to be omitted.

The tumours used by the GOLPH3 cluster of oncogenes which can be advantageously treated with GSL metabolism inhibitors comprise liver, lung, prostate, ovarian and colon cancers and melanomas.

Examples of GSL metabolism inhibitors comprise iminosugars such as miglustat, ceramide analogues such as eliglustat or the tartrate thereof, venglustat, or N-[(1R,2R)-2-(2,3-dihydro-1,4-benzodioxin-6-yl)-2-hydroxy-1-(1-pyrrolidinylmethyl)ethyl]-nonanamide (GENZ-123346), inhibitors of P-GP, the flippase necessary for metabolism of GSLs, such as zosuquidar and tamoxifen derivatives as the metabolite thereof. Miglustat and eliglustat are medicaments already in use, whereas venglustat is currently undergoing first-level clinical validation. Said compounds are collectively referred to hereafter as GSL metabolism inhibitors.

The table below lists other compounds with known inhibitory activity against the human enzyme glucosylceramide synthase (GCS) used in scientific experiments and under evaluation together with compounds having inhibitory activity against P-GP, and therefore against GSL metabolism. The right-hand column cites one of the numerous references available in the scientific literature.

| Acronym/class of | |
|---|---|
| compounds | REF |
| T-036 | DOI: 10.1111/jnc.15492 |
| EXEL-0346 | dx.doi.org/10.1021/jm300122u |
| Genz-123346 | doi: 10.1016/j.jbc.2021.100470 |
| Genz-682452 | |
| BZ1 | DOI: 10.1016/j.nbd.2021.105507 |
| GZ667161 | https://journals.plos.org/plosone/article?id=10.1371/journal.pone.0043310 |
| Deoxygalactonojirimycin | doi: 10.1021/ml200050s |
| Deoxynojirimycin | |
| Isofagomine | doi: 10.1002/cbic.200800249 |
| Pyrimethamine | |
| Deoxynojirimycin | Future Med. Chem. (2013) 5(5), 573-590 |
| derivatives | |
| Iminoxylitol derivatives | Future Med. Chem. (2013) 5(5), 573-590 |
| Lipophilic DIX | Future Med. Chem. (2013) 5(5), 573-590 |
| derivatives | |
| D-lactams and N- | Future Med. Chem. (2013) 5(5), 573-590 |
| substituted ε- | |
| hexonolactams | |
| Analogues of bicyclic | Future Med. Chem. (2013) 5(5), 573-590 |
| sp2-iminosugars | |
| Noeurostegine | Future Med. Chem. (2013) 5(5), 573-590 |
| Calystegine B2 | Future Med. Chem. (2013) 5(5), 573-590 |
| Isofagomine derivatives | Future Med. Chem. (2013) 5(5), 573-590 |
| Inosamine derivatives | Future Med. Chem. (2013) 5(5), 573-590 |
| Amino- and diamino- | Future Med. Chem. (2013) 5(5), 573-590 |
| cyclitols | |
| Polyhydroxylated | Future Med. Chem. (2013) 5(5), 573-590 |
| bicyclic isoureas and | |
| guanidines | |
| Pyrazolopyrimidine | Future Med. Chem. (2013) 5(5), 573-590 |
| derivatives | |
| ABX | Future Med. Chem. (2013) 5(5), 573-590 |
| 2-S-alkylsulphanyl-1,3- | Future Med. Chem. (2013) 5(5), 573-590 |
| oxazoline | |
| d-glucopyranose | |
| derivatives | |
| D-threo-4*-hydroxy-P4 | THE JOURNAL OF BIOLOGICAL CHEMISTRY Vol. |
| | 274, No. 21, Issue of May 21, pp. 14662-14669, 1999 |
| Benzofuran-carboxamide | ACS Med. Chem. Lett. 2022, 13, 879-880 |
| compounds | |
| Amino-myo-inositol | dx.doi.org/10.1021/jm300342q |
| derivatives | |
| 2,6-substituted 4- | www.pnas.orgcgidoi10.1073pnas.0705637104 |
| methylquinoline | |
| N-aryl-4- | www.pnas.orgcgidoi10.1073pnas.0705637104 |
| (arylsulphonamido) | |
| benzamide compounds | |
| 2-(4,6-substituted-1,3,5- | www.pnas.orgcgidoi10.1073pnas.0705637104 |
| triazin-2-ylamino)ethanol | |
| analogues | |

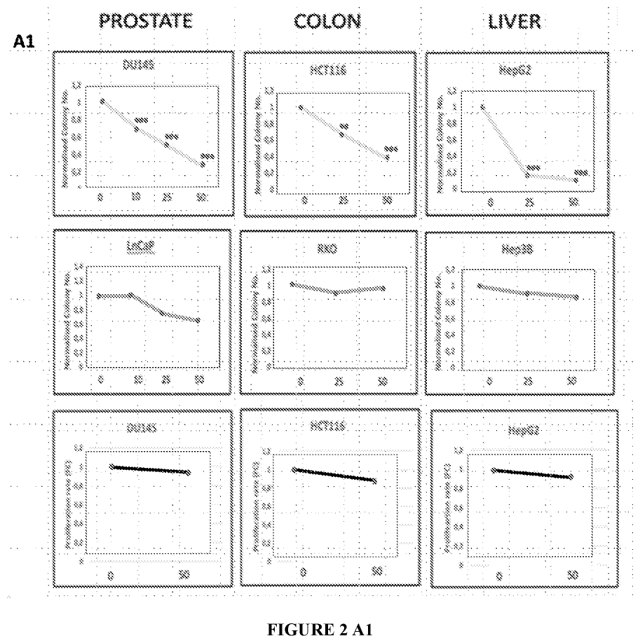

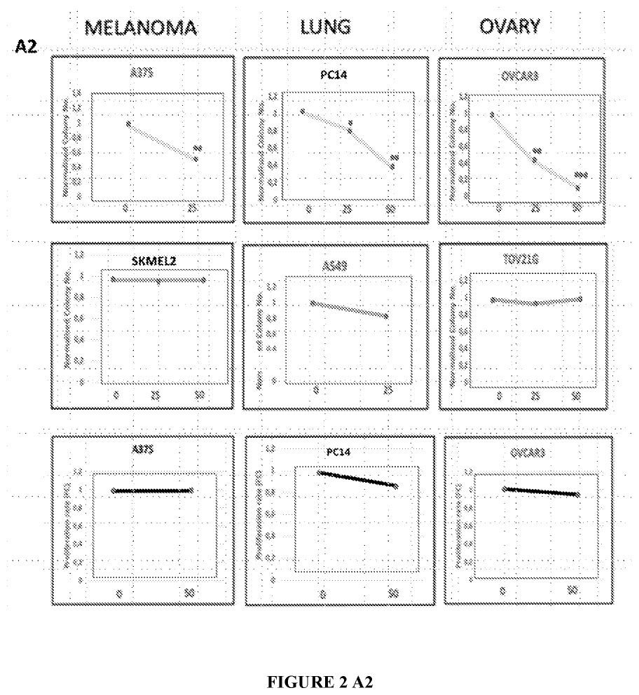

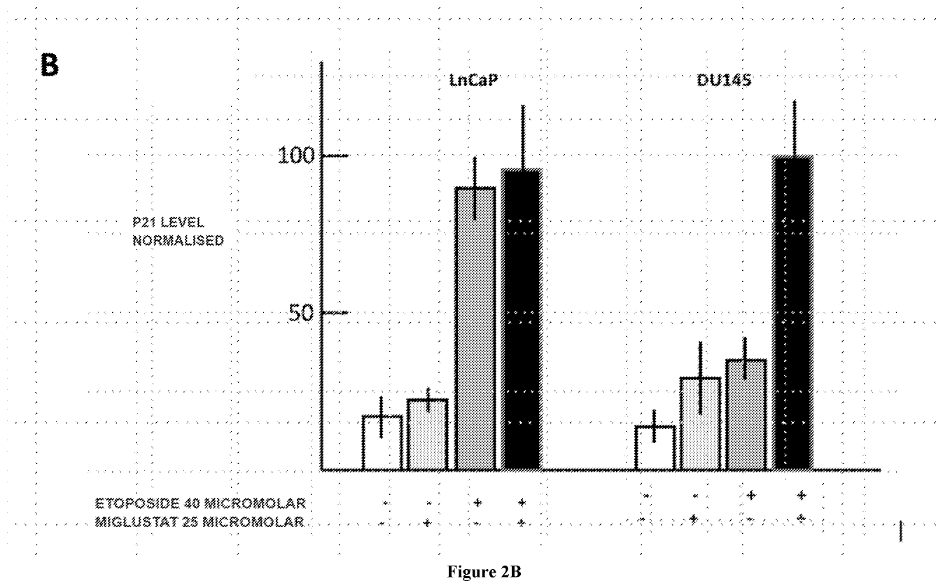

FIGS. 2A1, 2A2 and 2B present the data, reported by way of example, in support of the conclusions set out above.

The GSL metabolism inhibitors listed above were used on tumour lines expressing an oncogene belonging to the GOLPH3 cluster. The results demonstrate suppression of the resistance induced by said oncogenes to apoptosis caused by loss of anchorage to the matrix and to chemotherapy agents (in this case etoposide).

The growth of tumour lines was evaluated under different experimental conditions. 12 tumour lines, divided into six pairs derived from six different types of tumour, were used. Each pair was formed by a tumour line expressing an oncogene belonging to the GOLPH3 cluster and by a line which is derived from the same type of tumour, but does not express said oncogenes. The growth of the lines was observed under 3 experimental conditions: 1) absence of anchorage to the matrix; 2) exposure to chemotherapy agents; 3) undisturbed growth under adhesion to the matrix.

FIG. 2A1 demonstrates that growth under anchorage-independent conditions of prostate, colon and liver tumour lines expressing an oncogene belonging to the GOLPH3 cluster is inhibited by miglustat (at concentrations ranging from 0 to 50 micromolar, similar to those known to inhibit the GCS enzyme in the cell) (top series, light grey), whereas growth under the same conditions of tumour lines deriving from the same tumour type which do not express GOLPH3 is not inhibited by miglustat (middle series, dark grey). Growth under conditions of adhesion to the matrix and absence of stress (bottom series, black) of the lines expressing an oncogene belonging to the GOLPH3 cluster is not inhibited by miglustat. Inhibition of GSL synthesis was about 70% (at 50 micromolar miglustat). Depletion of the members of the GOLPH3 cluster by siRNA 25 nanomolar targeting the oncogene belonging to the GOLPH3 cluster expressed in the line in question reached 90%, and inhibited growth more markedly than miglustat at the concentrations described (not shown).

FIG. 2A2 presents the same results as FIG. 2A1, but in melanoma, lung cancer and ovarian cancer cell lines.

FIG. 2B shows the growth of tumour lines under adhesion to the matrix in the presence of chemotherapy agents (chemoresistance). The lines not expressing an oncogene belonging to the GOLPH3 cluster (LnCap) treated with the chemotherapy agent etoposide exhibited cell cycle arrest (strong increase in p21 synthesis), and co-treatment with eliglustat does not modify said situation. Conversely, the line expressing an oncogene belonging to the GOLPH3 cluster (DU145) is resistant to etoposide, but loses said resistance in the presence of eliglustat. Eliglustat alone has no effect. The same kind of result is obtained by using the P-GP inhibitor zosuquidar.

### DESCRIPTION OF METHODS

### Reagents

### Cell Lines and Culture Conditions:

Ovarian cancer NIHOVCAR3 and TOV21G, liver cancer HepG2 and Hep3B, cutaneous melanoma A375 and SKMEL2, colon cancer HCT116 and RKO, prostate cancer DU145 and LnCaP and lung cancer A549 and PC14 cell lines were purchased from the American Tissue Type Collection (ATTC, USA). Cell lines DU145, A375, A549, HCT116 and HepG2 were cultured in DMEM supplemented with 10% FBS, 2 mM of L-glutamine and 100 U/ml of penicillin and streptomycin; SKMEL5, Hep3B and RKO were cultured in MEM supplemented with 10% FBS, 1 mM of non-essential amino acids (NAA), 2 mM of L-glutamine, 100 U/ml of penicillin and streptomycin; while cell lines NIHOVCAR3, TOV21G, LnCaP and PC14 were cultured in RPMI supplemented with 10% FBS, 2 mM of L-glutamine and 100 U/ml of penicillin and streptomycin. All the tumour cell lines used in the study were tested and confirmed to be free of mycoplasma, and cultured at a controlled temperature (37° C.) and atmosphere (5% CO2 and 95% air).

Medicaments: Miglustat (from Cayman), Eliglustat (from Cayman), Genz-123346 (from Sigma), Etoposide (from Sigma), Zosuquidar (from SelleckChem)

### Small RNA Interference (RNAi):

| | |
|---|---|
| GOLPH3 | NM_022130 |
| B4GALT5 (LCS) | NM_004776 |

### Primary and Secondary Antibodies:

| ANTIBODY | Source | IDENTIFIER |
|---|---|---|
| Polyclonal Rabbit | Abcam | Cat # ab98023 |
| GOLPH3 | | RRID: AB_10860828 |
| Polyclonal Mouse | Abcam | Cat # ab69171 |
| GOLPH3 | | RRID: AB_2279272 |
| Monoclonal Mouse | Santa Cruz | Clone 6C5 |
| GAPDH | Biotechnology | Cat #sc-32233 |
| | | RRID: AB_627679 |
| Monoclonal Mouse anti-β | Sigma | Clone AC-74 |
| Actin | | Cat #A2228 |
| | | RRID: AB_476697 |
| Rabbit monoclonal | Cell Signalling | Clone 12D1 |
| antibody p21 Waf1/Cip1 | Technology | Cat #2947 |
| | | RRID: AB_823586 |
| Polyclonal Rabbit PARP | Cell Signalling | Clone 46D11 |
| | Technology | Cat #9532 |
| Anti-rabbit, goat IgG H & | Millipore | Cat # 401315 |
| L peroxidase conjugated | | |
| Anti-mouse, goat IgG H & | Millipore | Cat # 401215 |
| L peroxidase conjugated | | |

### Treatments

Gene Silencing by siRNA

The siRNAs used in this study were obtained from Sigma-Aldrich (Table 1). Oligofectamine or RNAi Max (Thermofisher Scientific, USA) was used for transfection of siRNA as previously described (Rizzo et al., 2021), at a concentration ranging between 5 and 50 nM. The silencing efficiencies were evaluated by Western blot or qPCR using specific primers.

### Blockade of GSL Synthesis by Inhibitors

GCS enzyme inhibitors such as miglustat, eliglustat and N-[(1R,2R)-2-(2,3-dihydro-1,4-benzodioxin-6-yl)-2-hydroxy-1-(1-pyrrolidinylmethyl)ethyl]-nonanamide (Genz-123346), or P-gp inhibitors such as zosuquidar, were used at two or three different concentrations, depending on the type of dose, to blockade glycosphingolipid biosynthesis in various tumour cells. For the growth assays, new media supplemented with the medicaments were added every three days.

Treatment with Chemotherapy Medicaments

The cells were treated with etoposide at a concentration ranging between 10 and 40 μM for 12-16 hours.

### Assays

### Cell Growth Assays

1. Growth in the absence of anchorage (anchorage-independent)
2. Growth of isolated cell clones (clonogenic analysis)
3. Growth under adhesion (under adhesion).

Both the anchorage-independent and clonogenic growth tests were conducted as described previously in Rizzo et al., EMBOJ, 2021. The growth and cell viability tests under adhesion were conducted by measuring XTT or ATP according to the manufacturer's protocol.

### Apoptosis Assay

As apoptosis activation index, cleavage of PARP by caspase 3 was evaluated by immunoblotting.

### Cell Cycle Arrest Assay

The p21 levels were determined by immunoblotting as cycle arrest index.

### Glycosphingolipid Metabolism

To analyse the glycosphingolipid metabolites we used a method based on radiolabelling of cell sphingosine followed by extraction of metabolites and separation by thin-layer chromatography (TLC) as previously described in Rizzo et al., EMOJ, 2021.

### Immunoblotting

The proteins were labelled with the primary antibodies mentioned (Table 2), and viewed using a secondary goat anti-rabbit or goat anti-mouse antibody IgG-HRP (Santa Cruz, USA) at a dilution of 1:10,000

### Statistics

The error bars correspond to the standard deviation (SD) or standard error (SEM) as indicated in the keys to the figures. The statistical evaluations were conducted with Student's “t” test *P<0.05, **P<0.01 and ***P<0.001 (ns, not significant).

### Mechanisms Underlying the Conclusions Described

### Growth in the Presence of Chemotherapy Agents (Chemoresistance)

Chemotherapy agents act by damaging the DNA of the target cell. This generates an apoptotic reaction, the function whereof is to prevent cells with severely damaged DNA from continuing to proliferate in the body. Said response is mediated by various mechanisms, the main ones being activation of p53 (the most extensively characterised) and increased ceramide, which causes the cell cycle arrest necessary to allow DNA repair and, if the damage is excessive, apoptosis, unless its effects are counteracted by oncogenes that directly promote survival and growth, or by mechanisms that rapidly consume the ceramide. GOLPH3 and the oncogenes belonging to the GOLPH3 cluster activate both of said protective mechanisms.

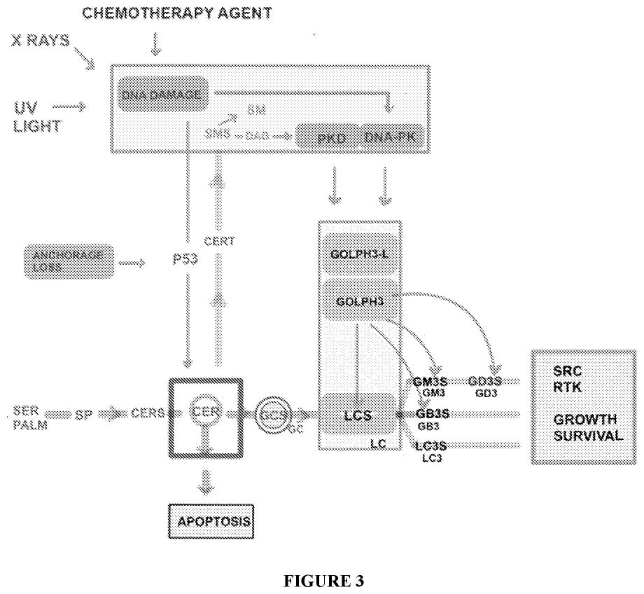

In addition, an important regulatory mechanism activates GOLPH3 specifically against stress conditions; DNA damage and increased ceramide activate mechanisms based on PKD and DNAPK kinases which phosphorylate and strongly potentiate the action of GOLPH3, and therefore ceramide consumption and production of pro-growth GSL. Said mechanism, under physiological conditions, acts as a control system that reduces the increase in ceramide and the consequent apoptotic effect induced by DNA damage which, unless moderated, may be uncontrolled and destructive in non-tumour cells. However, in tumour cells overexpressing GOLPH3, said system induces excessive suppression of apoptotic signals and an excessive increase in growth signals which, together, lead to chemoresistance and increased tumour proliferation, making said tumour cells “invulnerable” to the apoptotic reaction induced by chemotherapy agents, according to the mechanism illustrated in FIG. 3.

Said regulatory mechanism in particular explains why the oncogene GOLPH3 is selectively effective in protecting tumour cells against apoptosis under stress conditions such as DNA damage wherein large amounts of ceramide are produced, namely because said oncogene represents a potent chemoresistance factor, whereas it is not very active in the absence of stress. It therefore also explains why GCS inhibitors such as eliglustat, which blockade ceramide consumption and synthesis of pro-growth GSL, eliminate chemoresistance due to the effect of GOLPH3 on GSLs, and its oncogenic action.

### Growth in the Absence of Anchorage

GOLPH3 also plays a protective role against apoptosis under other stress conditions such as loss of anchorage to the matrix, wherein strong production of ceramide takes place. Though less characterised, the apoptosis mechanism due to loss of anchorage and the apoptosis resistance mechanism mediated by GOLPH3 under said conditions are similar to those described above for chemoresistance. The same conclusions also apply to the other oncogenes belonging to the GOLPH3 cluster.

The scheme in FIG. 3 shows the regulatory mechanisms based on PKD and DNAPK which potentiate the ability of GOLPH3 to induce resistance to DNA damage and loss of anchorage to the matrix, and the effect of GCS inhibitors as suppressors of said properties of GOLPH3. DNA damage (top rectangle, against grey background, on left) induces a strong increase in transcription of ceramide synthase and therefore of ceramide production. Part of the ceramide is used for production of sphingomyelin (SM) and diacylglycerol (DAG), which then activates PKD kinase (top rectangle, against grey background, on right). Said kinase phosphorylates GOLPH3 and potentiates its activity. A second effect of DNA damage is direct activation of DNAPK kinase (top rectangle, against grey background, on right), which has a similar effect to that of PKD, and further potentiates the action of GOLPH3. GOLPH3 is therefore specifically potentiated during the event involving DNA damage (or loss of anchorage with consequent ceramide production). This makes the action of GOLPH3 particularly efficient under said conditions of severe stress.

GCS inhibitors such as eliglustat, or P-GP inhibitors such as zosuquidar, blockade both ceramide consumption by GCS and successive enzymes, and synthesis of pro-growth GSLs, and therefore blockade the antiapoptotic and pro-growth action of GOLPH3.

For the therapeutic uses intended according to the invention, GCS or P-GP inhibitors can be formulated in compositions suitable for oral or parenteral administration, using conventional techniques. The doses will depend on the type and stage of the disorder and the patient's weight, sex and age, and can be determined on the basis of pre-clinical and clinical trials. In the case of miglustat, eliglustat and other inhibitors already in clinical use, the oral doses broadly correspond to those already used for the known, authorised therapeutic indications.

The activity of GCS and P-GP inhibitors in inhibiting the proliferation of tumour cells dependent on the GOLPH3 cluster of oncogenes was demonstrated using lung, ovary, prostate, skin (melanoma) and colon cell lines.

1. A method of treating GOLPH 3 and GOLPH3-cluster oncogene expression-dependent and chemoresistant tumours in a subject in need thereof with a glycosphingolipid metabolism inhibitor, said method comprising
treating said subject with a pharmaceutical effective amount of said glycosphingolipid metabolism inhibitor.

2. The method according to claim 1, wherein said glycosphingolipid metabolism inhibitor for use according to claim 1 which is an iminosugar.

3. The method according to claim 2 wherein the iminosugar is miglustat.

4. The method according to claim 1, wherein said glycosphingolipid metabolism inhibitor is selected from N-[(1R,2R)-2-(2,3-dihydro-1,4-benzodioxin-6-yl)-2-hydroxy-1-(1-pyrrolidinyl-methyl)ethyl]-nonanamide, eliglustat, eliglustat tartrate, venglustat and zosuquidar.

5. The method according to claim 1, wherein the GOLPH 3 and GOLPH3-cluster oncogene expression-dependent and chemoresistant tumours are liver, lung, prostate, ovarian and colon cancers and melanomas.

6. A method for classifying a tumour as sensitive or resistant to treatment with a glycosphingolipid inhibitor comprising measuring the GOLPH3 and GOLPH3-cluster expression level.

7. The method according to claim 6 wherein the tumour is chemoresistant.

8. Method for identifying tumours responding to glycosphingolipid metabolism inhibitors in a subject in need thereof, said method comprising
detecting of genomic amplification of GOLPH3 in said tumors of said subject.
